(19) United States
(12) Patent Application Publication
(10) Pub. No.: US 2009/0004645 A1
(43) Pub. Date: Jan. 1, 2009

(54) Methods for Identifying Genes that Mediate a Response of a Living Cell to an Agent

(75) Inventors: Mao Mao, Redmond, WA (US); Aimee Jackson, Seattle, WA (US); Peter S. Linskey, Seattle, WA (US); Hongyue Dai, Kenmore, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Rosetta Inpharmatics LLC, Seattle, WA (US)

(21) Appl. No.: 11/578,216

(22) PCT Filed: Apr. 12, 2005
(86) PCT No.: PCT/US2005/012452

§ 371 Date: Aug. 18, 2008

Related U.S. Application Data
(60) Provisional application No. 60/562,392, filed on Apr. 15, 2004.

(51) Int. Cl. C12Q 1/68

(52) U.S. Cl. 435/6

(57) ABSTRACT

In one aspect, the present invention provides methods for determining whether a gene mediates the response of a living cell to an agent. In another aspect, the present invention provides methods to identify a mammalian subject responsive to a KSP inhibitor.

### FIELD OF THE INVENTION

The present invention relates to methods for identifying genes that mediate a response of a living cell to an agent, such as a response of a living cell to therapeutic drug molecules.

### BACKGROUND OF THE INVENTION

Identification of a gene that mediates the response of a living cell to an agent permits the development of compositions and methods for making desirable changes in the response of the cell to the agent. For example, one approach to developing new treatments for a disease in a mammal is to first identify a gene, expressed in a mammal, that contributes to the development and/or maintenance of the disease. Such a gene may, for example, be overexpressed, underexpressed, and/or expressed at an incorrect location or time compared to the normal expression pattern and expression level of the gene. Again by way of example, a mutated gene may express a variant of its normal gene product (e.g., protein, or biologically active RNA molecule), and the variant gene product may inappropriately affect one or more biological processes. For example, a mutated gene may encode a variant receptor protein that is expressed in a mammalian cell type, thereby causing the constitutive activation of one or more biochemical signal transduction pathways associated with the receptor. The constitutively activated pathways may cause the development of a disease in the affected mammal.

Thus, a mutated gene product, or a gene product that is expressed at an inappropriate level, time, or place, or which has an expression pattern that is in any way abnormal, is potentially a target for drug therapy using molecules that interact with the gene product and inhibit its biological activity. Identifying and testing new drug molecules is a time consuming and expensive process, and so it is important to first correctly identify genes that contribute to the development and/or maintenance of a mammalian disease before attempting to identify molecules that interact with the gene products and inhibit their biological activity. Thus, there is a continuing need for methods to determine whether a gene, through the action of its encoded product, mediates the response of a living cell to a chemical agent. More generally, there is a continuing need for methods to determine whether a gene mediates the response of a living cell to an agent that may be chemical (e.g., a therapeutic drug), energetic (e.g., ultraviolet radiation), or physical (e.g., a physical force, such as the shear forces exerted on the cells of blood vessels).

### SUMMARY OF THE INVENTION

In a first aspect, the present invention provides methods for determining whether a gene mediates a response of a living cell to an agent, the method comprising the steps of: (a) functionally inactivating a gene in a living cell of a cell type by introducing interfering RNA molecules into the cell, wherein the gene has an expression pattern that correlates with a response of living cells of the cell type to an agent, and wherein a copy of the gene is present in all living cells of the cell type; (b) contacting the living cell, comprising the interfering RNA molecules, with the agent; and (c) determining whether the response of the living cell to the agent is different from the response of a living cell, that does not comprise the interfering RNA molecules, of the cell type to the agent, thereby determining whether the gene mediates the response of living cells of the cell type to the agent. The methods of this aspect of the invention optionally include the step of identifying the gene, that has an expression pattern that correlates with a response of the living cells of the cell type to an agent, before functionally inactivating the gene using interfering RNA molecules.

The methods of the first aspect of the invention are useful for determining whether any gene mediates any response of any living cell to any agent. For example, the methods of the first aspect of the invention can be used to determine whether a gene mediates (through its encoded product) a beneficial or adverse response of a living mammalian cell to a therapeutic drug. The product (e.g., protein) encoded by a gene that mediates a beneficial, or adverse, response of a living mammalian cell to a therapeutic drug is itself a potential target for a drug adapted to augment the effectiveness (or reduce the adverse side effects) of the therapeutic drug. For example, the methods of the first aspect of the invention can be used to determine whether a gene mediates resistance of mammalian cancer cells to a chemotherapeutic drug (i.e., the cancer cells become less sensitive to the drug after repeated exposure to the drug). The product (typically a protein) encoded by a gene that mediates resistance of cancer cells to a chemotherapeutic drug is a possible target for a drug that inhibits the activity of the gene product in cancer cells, thereby reducing the tendency of the cancer cells to become resistant to the chemotherapeutic drug. Again by way of example, the methods of the first aspect of the present invention can be used to identify genes that mediate a beneficial response (e.g., amelioration of a disease state) of a living mammalian cell, tissue, organ, and/or organism, to a therapeutic drug. The gene product is a possible target for a new drug that can be used to further ameliorate the disease state. Thus, the methods of the first aspect of the present invention can be used, for example, to contribute to the development of new drugs for treating a disease, or new drugs that increase the effectiveness of known drugs for treating a disease in humans and/or other mammals.

In another aspect the present invention provides methods for confirming that a gene contributes to a response of a living cell to an agent. The methods of this aspect of the invention each include the steps of (a) functionally inactivating a gene in a living cell of a cell type by introducing interfering RNA molecules into the cell, wherein the gene has an expression pattern that correlates with a response of living cells of the cell type to an agent, and wherein a copy of the gene is present in all living cells of the cell type; and (b) confirming that the gene contributes to the response of the living cell to the agent by determining that the response of the living cell to the agent is different from the response of a living cell, that does not comprise the interfering RNA molecules, of the cell type to the agent. The methods of this aspect of the invention can be used to confirm that any gene contributes to any response of a living cell to any agent. For example, the methods of this aspect of the invention can be used to confirm that a gene contributes to a desirable or adverse response of a living mammalian cell to a therapeutic drug.

In a further aspect, the present invention provides methods for identifying a mammalian subject responsive to a KSP inhibitor. These methods each include the steps of analyzing chromosome 20 from cancerous cells of a mammalian subject to determine whether portion 20q of chromosome 20 is amplified in the cancerous cells, wherein the mammalian subject is identified as being responsive to a KSP inhibitor provided that portion 20q of chromosome 20 is not amplified in the cancerous cells. Thus, the methods of this aspect of the invention are useful, for example, to identify human cancer patients who are likely to benefit from treatment with a KSP inhibitor (i.e., those human cancer patients identified as being responsive to a KSP inhibitor would likely be candidates for treatment with a KSP inhibitor).

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al. (1989) Molecular Cloning: A Laboratory Manual, 2nd ed., Cold Spring Harbor Press, Plainsview, N.Y. (1989), and Ausubel et al., Current Protocols in Molecular Biology (Supplement 47), John Wiley & Sons, New York (1999), for definitions and terms of the art.

As used herein, the term “agent” encompasses any physical, chemical, or energetic agent that induces a biological response in a living cell in vivo and/or in vitro. Thus, for example, the term “agent” encompasses chemical molecules, such as candidate therapeutic molecules that may be useful for treating one or more diseases in a living organism, such as in a mammal (e.g., a human being). The term “agent” also encompasses energetic stimuli, such as ultraviolet light. The term “agent” also encompasses physical stimuli such as forces applied to living cells (e.g., pressure, stretching or shear forces). The term “agent” also encompasses viruses capable of infecting a living cell. The term “agent” encompasses inhibitors of a KSP protein. Representative KSP inhibitors include small molecule organic compounds, such as semicarbazones and thiosemicarbazones. For example, the KSP inhibitor may be an aryl thiosemicarbazone. Exemplary aryl thiosemicarbazone KSP inhibitors include, but are not limited to, 1,1′-biphenyl-4-carbaldehyde thiosemicarbazone, 4-isopropylbenzaldehyde thiosemicarbazone (see, e.g., U.S. Pat. No. 3,849,575), 4-cyclohexylbenzaldehyde thiosemicarbazone, and 4isopropyl-3-nitrobenzaldehyde thiosemicarbazone ( see, e.g., Saripinar et al. (1996) Arzneimittel-Forschung 46(II):824-8). Other exemplary KSP inhibitors are described in the following published international patent applications which are each incorporated herein by reference: WO 2003/106417A1, WO 2003/105855A1, WO 2003/099211A2, WO 2003/079973A2, WO 2003/050122A2, WO 2003/050064A2, WO 2003/049679A2, WO 2003/049678A2, WO 2003/049527A2, and WO 2003/039460A2.

KSP is an abbreviation for kinesin spindle protein. KSPs are sometimes referred to in the literature as hsKSP, KNSL-1, or kinesin-like-1. KSPs are members of the BimC kinesin subfamily, and are mitotic kinesin proteins that are essential for mitosis in cells. Kinesins are enzymes that translate energy released by hydrolysis of adenosine triphosphate (ATP) into mechanical force along intracellular filaments in order to carry out work within a cell. Mitotic kinesins are a functional subgroup of kinesins that work in an ordered fashion to facilitate mitosis, the division of DNA during cell division. These cytoskeletal motor proteins act in concert in a mechanical cascade to effect mitotic spindle formation and function. KSPs are reviewed in Miki et al., Proc. Nat'l. Acad. Sci. 98:7004-7011 (2001), and in Dagenbach and Endow, J. Cell Sci. 117:3-7 (2004). Typically KSPs are at least 90% identical to the representative KSP amino acid sequence set forth in SEQ ID NO:1.

STK6 is an abbreviation for serine/threonine kinase 6 which is a member of the Aurora kinase family. Members of the Aurora kinase family contribute to the regulation of chromosome segregation and cytokinesis during mitosis. STK 6 proteins are described, for example, in Kimura, M.; et al., J. Biol. Chem. 272: 13766-13771 (1997), and in Kimura, M., et al., Cytogenet. Cell Genet. 79: 201-203 (1997). Typically genes that encode an STK 6 protein are at least 90% identical to the representative STK 6 gene sequence set forth in SEQ ID NO:2. Typically genes that encode an STK 6 protein hybridize to the complement of the representative STK 6 gene sequence set forth in SEQ ID NO:2 under conditions of 5×SSC at 55° C. for 12 hours, followed by washing in 5×SSC at 55° C. for 1 hour. Some genes that encode an STK 6 protein hybridize to the complement of the representative STK 6 gene sequence set forth in SEQ ID NO:2 under conditions of 5×SSC at 65° C. for 12 hours, followed by washing in 5×SSC at 65° C. for 1 hour. A representative hybridization protocol is set forth at pages 9.52-9.55 of Sambrook et al., supra.

TPX 2 proteins contribute to the formation of microtubules associated with chromosomes during mitosis (see, e.g., Gruss, et al., Nature Cell. Biol. 4: 871-879 (2002), Heidebrecht, H. J. et al., Blood 90: 226-233 (1997), Kufer, T. A., J. Cell Biol. 158: 617-623 (2002), Manda, R., et al., Genomics 61: 5-14 (1999) and Zhang, Y., et al., Cytogenet. Cell Genet. 84: 182-183 (1999)). Typically genes that encode a TPX 2 protein are at least 90% identical to the representative TPX 2 gene sequence set forth in SEQ ID NO:3. Typically genes that encode a TPX 2 protein hybridize to the complement of the representative TPX 2 gene sequence set forth in SEQ ID NO:3 under conditions of 5×SSC at 55° C. for 12 hours, followed by washing in 5×SSC at 55° C. for 1 hour. Some genes that encode a TPX 2 protein hybridize to the complement of the representative TPX 2 gene sequence set forth in SEQ ID NO:3 under conditions of 5×SSC at 65° C. for 12 hours, followed by washing in 5×SSC at 65° C. for 1 hour. A representative hybridization protocol is set forth at pages 9.52-9.55 of Sambrook et al., supra.

The term “percent identity” or “percent identical”, is the percentage of amino acid residues in a candidate polypeptide sequence that are identical with a subject polypeptide molecule sequence, or the percentage of nucleic acid residues in a candidate nucleic sequence that are identical with a subject nucleic acid molecule sequence, after aligning the candidate and subject sequences to achieve the maximum percent identity. When making the comparison, no gaps are introduced into the candidate sequence in order to achieve the best alignment with the subject sequence.

Sequence identity can be determined, for example, by using computer programs for determining similarity, such as TBLASTN, BLASTP, FASTA, TFASTA, and CLUSTALW (Pearson and Lipman, 1988, Proc. Natl. Acad. Sci. U.S.A. 85:2444-2448; Altschul et al., 1990, J. Mol. Biol. 215:403-410; Thompson et al., 1994, Nucleic Acids Res. 22:4673-4680; and Higgins et al., 1996, Methods Enzymol. 266:383402). Specifically, Basic Local Alignment Search Tool (BLAST) (Altschul et al., 1990, J. Mol. Biol. 215:403410, “The BLAST Algorithm”; Altschul et al., 1997, Nucl. Acids Res. 25:3389-3402) is a heuristic search algorithm tailored to searching for sequence similarity which ascribes significance using the statistical methods of Karlin and Altschul (1990, Proc. Nat'l Acad. Sci. U.S.A., 87:2264-2268; 1993, Proc. Nat'l Acad. Sci. U.S.A. 90:5873-5877). Five specific BLAST programs are used to perform the following tasks: (1) the WU-BLASTP program compares an amino acid query sequence against a protein sequence database; (2) the WU-BLASTN program compares a nucleotide query sequence against a nucleotide sequence database; (3) the BLASTX program compares the six-frame conceptual translation products of a nucleotide query sequence (both strands) against a protein sequence database; (4) the TBLASTN program compares a protein query sequence against a nucleotide sequence database translated in all six reading frames (both strands); and (5) the TBLASTX program compares the six-frame translations of a nucleotide query sequence against the six-frame translations of a nucleotide sequence database. The foregoing BLAST tools are available at the website of the National Center for Biotechnology Information, National Library of Medicine, Building 38A, Bethesda, Md. 20894, U.S.A.

Smith-Waterman (Smith-Waterman, 1981, J. Mol. Biol. 147:195-197) is a mathematically rigorous algorithm for sequence alignments. FASTA (see Pearson et al., 1988, Proc. Nat'l Acad. Sci. U.S.A., 85:2444-2448) is a heuristic approximation to the Smith-Waterman algorithm. For a general discussion of the procedure and benefits of the BLAST, Smith-Waterman and FASTA algorithms see Nicholas et al., 1998, “A Tutorial on Searching Sequence Databases and Sequence Scoring Methods” and references cited therein.

First Aspect of the Invention: In a first aspect, the present invention provides methods for determining whether a gene mediates a response of a living cell to an agent, the method comprising the steps of: (a) functionally inactivating a gene in a living cell of a cell type by introducing interfering RNA molecules into the cell, wherein the gene has an expression pattern that correlates with a response of living cells of the cell type to an agent, and wherein a copy of the gene is present in all living cells of the cell type; (b) contacting the living cell, comprising the interfering RNA molecules, with the agent; and (c) determining whether the response of the living cell to the agent is different from the response of a living cell, that does not comprise the interfering RNA molecules, of the cell type to the agent, thereby determining whether the gene mediates the response of living cells of the cell type to the agent. The methods of this aspect of the invention optionally include the step of identifying the gene, that has an expression pattern that correlates with a response of the living cells of the cell type to an agent, before functionally inactivating the gene using interfering RNA molecules.

The methods of the first aspect of the present invention can be practiced using living cells in vivo, or using cultured cells in vitro. It will be understood that typically a multiplicity of living cells (e.g., a population of cells cultured in vitro, such as a cultured animal tissue comprising a multiplicity of cells, or such as a culture of individual cells) are treated in accordance with the methods of the first aspect of the present invention.

Any cell type can be used in the practice of the present invention, including cell types obtained from microorganisms, insects such as Drosophila, other invertebrates such as nematodes, plants, mammals, humans, non-human primates, cats, dogs, and farm animals (e.g., cows). Any type of mammalian cancer cells can be used in the practice of this aspect of the invention.

In the context of this aspect of the invention, a gene mediates a response of a living cell to an agent if a response of a living cell to an agent depends, in whole or in part, on the product encoded by the gene. Thus, a response of the cell to the agent either does not occur in the absence of the gene product, or the magnitude and/or duration of the response is reduced in the absence of the gene product compared to the magnitude and/or duration of the response in the presence of the gene product. A gene product includes proteins, peptides, and nucleic acid molecules (e.g., any type of RNA molecule).

The response of a living cell to an agent can be any type of response including, for example, an increase or a decrease in the level, intensity and/or duration of a biochemical or physiological response, or, for example, any change in a gene expression pattern.

A gene that is functionally inactivated in the practice of this aspect of the invention has an expression pattern that correlates with a response of living cells of a cell type to an agent. The correlation must be statistically significant, and can be a positive correlation, or a negative correlation.

Functional Inactivation Using Interfering RNA Molecules: In the practice of the methods of this aspect of the invention, a gene is functionally inactivated in a living cell of a cell type by introducing interfering RNA molecules into the cell. As used in this context, “functional inactivation” of a gene means completely destroying, or partially destroying, RNA transcribed from the gene, and/or completely preventing, or partially preventing, translation of RNA transcribed from the gene. Thus, the function of the gene is substantially abolished, or completely abolished, by the interfering RNA molecules that are introduced into the cell.

Interfering RNA molecules are double-stranded RNA molecules that are completely complementary (or substantially complementary) to all, or a portion, of a messenger RNA (mRNA) in a living cell, and that promote the degradation of the mRNA through the process of RNA interference (RNAi). RNA interference is a naturally-occurring phenomenon that is believed to function in protecting cells from invasion by RNA viruses. While not wishing to be bound by theory, it is currently believed that, in the process of RNA interference, when a cell is infected by a double stranded RNA virus (abbreviated as dsRNA virus), the dsRNA is recognized and targeted for cleavage by an RNaseIII-type enzyme termed Dicer. The Dicer enzyme “dices” the RNA into short duplexes of 21 nucleotides, termed siRNAs or short-interfering RNAs, composed of 19 nt of perfectly paired ribonucleotides with two unpaired nucleotides on the 3′ end of each strand. These short duplexes associate with a multiprotein complex termed RISC, and direct this complex to mRNA transcripts with sequence similarity to the siRNA. As a result, nucleases present in the RISC complex cleave the mRNA transcript, thereby abolishing expression of the gene product. In the case of viral infection, this mechanism results in destruction of viral transcripts, thus preventing viral synthesis.

Since the siRNAs are double-stranded, either strand has the potential to associate with RISC and direct silencing of transcripts with sequence similarity. RNA interference acts upon any exogenous, double-stranded, RNA molecule, and is not restricted to acting upon the genomes of RNA viruses.

The class of interfering RNA molecules includes short interfering RNAs (siRNAs), short hairpin RNAs (shRNAs), and long double stranded RNAs, and chemically-modified derivatives of any of the foregoing types of interfering RNA molecules. Short interfering RNAs (siRNAs) are double-stranded RNA molecules, 21-23 nucleotides long, that mediate the degradation of messenger RNA (mRNA) molecules, thereby functionally inactivating the gene(s) that encodes the degraded mRNA molecules. The mRNA molecules that are degraded include a nucleic acid sequence that is complementary (or at least partially complementary) to one of the two strands of the siRNA molecule. Some siRNA molecules include 9, 10, 11, 12, 13, 14, 15, 16, 17, 18 or 19 contiguous nucleic acid residues that form a sequence of nucleic acid residues that is complementary to a portion of a target mRNA molecule.

siRNA molecules typically, although not necessarily, include a 19-nucleotide region that is completely-complementary (or at least partially complementary) to a 19-nucleotide region of a target RNA molecule, and a 2-nucleotide 3′ overhang at each end of the 19-nucleotide region. Custom siRNA molecules having defined nucleic acid sequences are commercially available, such as the On-Target™ siRNA product, or the SMART™ siRNA product, sold by Dharmacon, Inc., 2650 Crescent Drive, #100, Lafayette, Colo. 80026.

A representative method for selecting an siRNA molecule useful for mediating the degradation of a target mRNA molecule is set forth in U.S. provisional patent application Ser. No. 60/515,180, filed on Oct. 27, 2003, which is incorporated herein by reference in its entirety. Representative methods for using siRNA molecules to promote degradation of target mRNA molecules in mammalian cells are set forth in U.S. provisional patent application Ser. No. 60/515,223, filed on Oct. 27, 2003, which is incorporated herein by reference in its entirety.

In the practice of the present invention synthetic siRNA molecules can be introduced into living cells, thereby mimicking the product of Dicer cleavage (see, e.g., Elbashir et al., Nature 411:494498, 2001; Elbashir et al., Genes Dev. 15:188-200, 2001, each of which publications is incorporated by reference herein in their entirety). siRNAs can be chemically synthesized, or, for example, can be produced in vitro by cleavage of double-stranded RNA by recombinant Dicer.

It has been shown that siRNA can be used to functionally inactivate genes in vivo. For example, Fas-mediated apoptosis is implicated in a broad spectrum of liver diseases, where lives could be saved by inhibiting apoptotic death of hepatocytes. Song (Song et al., Nat. Medicine 9:347-351, 2003) injected mice intravenously with siRNA targeted S to the Fas receptor. The Fas gene was silenced in mouse hepatocytes at the mRNA and protein levels, thereby preventing apoptosis, and protecting the mice from hepatitis-induced liver damage. As another example, Sorensen et al. (J. Mol. Biol. 327:761-766 (2003)) injected mice intraperitoneally with siRNA targeting TNF-α. Lipopolysaccharide-induced TNF-α gene expression was inhibited, and these mice were protected from sepsis.

Short hairpin RNAs (shRNAs) are short RNA molecules wherein two complementary portions of the same RNA molecule hybridize to each other to form a double-stranded stem (typically of 19 to 29 base pairs) and a single-stranded loop. When the shRNA is introduced into a living cell the double-stranded stem is processed to leave a short, double-stranded, RNA molecule consisting of from 19 to 23 bp. For example, a desired shRNA sequence can be expressed from a vector (e.g., plasmid or virus) and includes an inverted repeat (that can hybridize with itself to form a hairpin structure) with an intervening sequence that forms a single-stranded loop when the inverted repeats hybridize together. The shRNA is introduced into a cell, or expressed in a cell from a vector, where it is processed by Dicer to remove the single-stranded loop, thereby producing an siRNA Plasmid-encoded shRNAs can be stably expressed in cells, allowing long-term functional inactivation of a gene in cells both in vitro and in vivo (e.g., in animals see, McCaffrey et al., Nature 418:38-39, 2002; Xia et al., Nat. Biotech. 20:1006-1010, 2002; Lewis et al., Nat. Genetics 32:107-108, 2002; Rubinson et al., Nat. Genetics 33:401-406, 2003; Tiscornia et al., Proc. Natl. Acad. Sci. USA 100:1844-1848, 2003, all of which are incorporated by reference herein in their entirety). The use of shRNAs to functionally inactivate genes is described in the following representative publications: Paddison et al., Genes Dev. 16:948-958 (2002); Brummelkamp et al., Science 296:550-553 (2002); Sui, G. et al., Proc. Natl. Acad. Sci. USA 99:5515-5520 (2002); Paddison, et al, Nature 428:427-431(2004); and Berns et al., Nature 428:431-437 (2004) all of which are incorporated by reference herein in their entirety.

Long double-stranded RNAs are double-stranded RNA molecules that are longer than 23 bp (typically longer than 100 bp), and that are processed within a living cell to produce one or more siRNA molecules.

Other representative methods for functionally inactivating a gene using interfering RNA molecules are described in the following, copending, United States provisional patent applications that are each incorporated herein by reference in their entirety: U.S. Provisional Patent Application Ser. No. 60/515,223, filed on Oct. 27, 2003; and U.S. Provisional Patent Application Ser. No. 60/515,180, filed on Oct. 27, 2003. Other representative methods for functionally inactivating a gene using interfering RNA molecules are described in the following patents and published patent applications that are each incorporated herein by reference in their entirety: U.S. Pat. No. 6,506,559; U.S. Patent Application Publication No. US 2002/0086356; PCT publication WO 02/44321; and PCT publication WO 03/006477.

Interfering RNA molecules (or vectors encoding interfering RNA molecules) can be introduced into cells by any useful method. For example, interfering RNA molecules (or vectors encoding interfering RNA molecules) can be introduced into mammalian cells by lipofection, using a lipid reagent such as lipofectamine or oligofectamine, or by electroporation. Again by way of example, interfering RNA molecules can be taken up spontaneously by Drosophila cells and nematodes (e.g., by adding the interfering RNA molecules to the Drosophila culture medium, or by soaking the nematodes in a culture medium that includes the interfering RNA molecules). Additionally, for example, vector-encoded shRNA can be cloned into E. coli which are mixed with the nematode food, and the interfering RNA molecules are taken up by cells within the nematode gut.

More generally, nucleic acid molecules can be introduced into cultures of mammalian cells, and other cells that do not have rigid cell walls, using the calcium phosphate method as originally described by Graham and Van der Eb (Virology, 52:546 [1978]) and modified as described in sections 16.32-16.37 of Sambrook et al., supra. Other exemplary methods for introducing DNA into cells include the use of Polybrene (Kawai and Nishizawa, Mol. Cell. Biol., 4:1172 [1984]), and electroporation (Neumann et al., EMBO J., 1:841 [1982]).

Many methods are known in the art for introducing nucleic acid molecules into plant cells. Representative examples include electroporation-facilitated DNA uptake by protoplasts in which an electrical pulse transiently permeabilizes cell membranes, permitting the uptake of a variety of biological molecules, including nucleic acid molecules (Rhodes et al., Science, 240:204-207 [1988]); treatment of protoplasts with polyethylene glycol (Lyznik et al., Plant Molecular Biology, 13:151-161 [1989])); and bombardment of cells with DNA-laden microprojectiles which are propelled by explosive force or compressed gas to penetrate the cell wall (Klein et al., Plant Physiol. 91:440-444 [1989] and Boynton et al., Science, 240:1534-1538 [1988]). Further, plant viruses can be used as vectors to transfer nucleic acid molecules to plant cells. Examples of plant viruses that can be used as vectors include the Cauliflower Mosaic Virus (Brisson et al., Nature 310: 511-514 (1984); Additionally, plant transformation strategies and techniques are reviewed in Birch, R. G., Ann Rev Plant Phys Plant Mol Biol, 48:297 (1997); Forester et al., Exp. Agric., 33:15-33 (1997).

Interfering RNA molecules can be delivered to an organ or tissue in an animal, such as a human, in vivo (see, e.g., Song et al., Nat. Medicine 9:347-351, 2003; Sorensen et al., J. Mol. Biol. 327:761-766, 2003; Lewis et al., Nat. Genetics 32:107-108, 2002, all of which are incorporated by reference herein in their entirety). For example, a solution of interfering RNA molecules can be injected intravenously into the animal, and thereafter be transported to an organ or tissue of interest where the interfering RNA molecules are taken up by cells of the organ or tissue.

Transcript Assays to Determine Gene Expression Patterns: the functionally inactivated gene has an expression pattern that correlates with a response of living cells of the cell type to an agent. In some embodiments of the invention, the expression pattern of the gene is known and does not have to be determined to practice the invention. In other embodiments, a gene having an expression pattern that correlates with a response of living cells of the cell type to an agent must be identified by analysing the expression pattern(s) of at least one gene (typically hundreds or thousands of genes) in response to an agent, and comparing the expression pattern(s) of the same gene(s) in the same type of cells that have not been contacted with the agent.

A gene useful in the practice of the first aspect of the present invention has an expression pattern that correlates with a response of a cell to an agent. Such genes can be identified, for example, by identifying different types of living cells (e.g., different types of mammalian cancer cells) that exhibit a particular response when contacted by an agent (e.g., the response is cell death when cells (e.g., cells cultured in vitro), are contacted with a chemical agent). Genes are identified that have an expression pattern in the untreated cells (that exhibit the response when treated with the agent), that is not observed in untreated cells that do not exhibit the response when treated with the agent.

Example 1 herein, provides examples of genes that are highly expressed in colon cancer cell lines that are also resistant to the KSP inhibitor L′962 (i.e., the level of expression of the genes is elevated in the cancer cell lines that exhibit the response (resistance) to L′962).

Again by way of example, a gene that has an expression pattern that correlates with a response of a cell to an agent can be a gene that has an expression pattern, in a cell contacted with an agent, that is statistically significantly different from the expression pattern of the same gene in the same cell type that has not been contacted with the same agent. The expression pattern comparison is made using cells of the same cell type, cultured under identical conditions, except that one cell (or population of cells) is contacted with the agent, and the other cell (or population of cells) is not contacted with the agent.

The expression level of a nucleotide sequence in a gene can be measured, for example, by any high throughput techniques. Irrespective of the method used to measure gene expression level, the result is either the absolute or relative amounts of transcripts or response data, including but not limited to values representing abundance ratios.

Measurement of the expression pattern can be made by hybridization to transcript arrays. For example, an expression pattern is obtained by hybridizing detectably labeled polynucleotides representing the nucleotide sequences in mRNA transcripts present in a cell (e.g., fluorescently labeled cDNA synthesized from total cell MRNA) to a microarray. A microarray is an array of positionally-addressable binding (e.g., hybridization) sites on a support for representing many of the nucleotide sequences in the genome of a cell or organism, preferably most or almost all of the genes. Each of such binding sites consists of polynucleotide probes bound to the predetermined region on the support. Microarrays can be made in a number of ways, of which several are described herein below. However produced, microarrays share certain characteristics. The arrays are reproducible, allowing multiple copies of a given array to be produced and easily compared with each other. Preferably, the microarrays are made from materials that are stable under binding (e.g., nucleic acid hybridization) conditions. The microarrays are preferably small, e.g., between about 1 cm² and 25 cm², preferably about 1 to 3 cm². However, both larger and smaller arrays are also contemplated and may be preferable, e.g., for simultaneously evaluating a very large number of different probes.

Preferably, a given binding site or unique set of binding sites in the microarray will specifically bind (e.g., hybridize) to a nucleotide sequence in a single gene (or transcript derived therefrom) from a cell or organism.

Microarrays include one or more probes, each of which has a polynucleotide sequence that is complementary to a subsequence of RNA or DNA to be detected. Each probe preferably has a different nucleic acid sequence, and the position of each probe on the solid surface of the array is preferably known. Indeed, the microarrays are preferably addressable arrays, more preferably positionally addressable arrays. More specifically, each probe of the array is preferably located at a known, predetermined position on the solid support such that the identity (i.e., the sequence) of each probe can be determined from its position on the array (i.e., on the support or surface). In some embodiments of the invention, the arrays are ordered arrays.

Preferably, the density of probes on a microarray or a set of microarrays is about 100 different (i.e., non-identical) probes per 1 cm² or higher. More preferably, a microarray has at least 550 probes per 1 cm², at least 1,000 probes per 1 cm², at least 1,500 probes per 1 cm² or at least 2,000 probes per 1 cm². In a particularly preferred embodiment, the microarray is a high density array, preferably having a density of at least about 2,500 different probes per 1 cm². The microarrays used in the invention therefore preferably contain at least 2,500, at least 5,000, at least 10,000, at least 15,000, at least 20,000, at least 25,000, at least 50,000 or at least 55,000 different (i.e., non-identical) probes.

In one embodiment, the microarray is an array in which each position represents a discrete binding site for a nucleotide sequence of a transcript encoded by a gene (e.g., for an exon of an mRNA or a cDNA derived therefrom). The collection of binding sites on a microarray contains sets of binding sites for a plurality of genes. For example, in various embodiments, microarrays can comprise binding sites for products encoded by fewer than 50% of the genes in the genome of an organism. Alternatively, microarrays can have binding sites for the products encoded by at least 50%, at least 75%, at least 85%, at least 90%, at least 95%, at least 99% or 100% of the genes in the genome of an organism. In other embodiments, microarrays can have binding sites for products encoded by fewer than 50%, by at least 50%, by at least 75%, by at least 85%, by at least 90%, by at least 95%, by at least 99% or by 100% of the genes expressed by a cell of an organism. The binding site can be a DNA or DNA analog to which a particular RNA can specifically hybridize. The DNA or DNA analog can be, e.g., a synthetic oligomer or a gene fragment, e.g. corresponding to a gene.

In some embodiments of the present invention, a gene or an exon in a gene is represented in the profiling arrays by a set of binding sites comprising probes with different polynucleotides that are complementary to different coding sequence segments of the gene or an exon of the gene. Such polynucleotides are preferably of the length of 15 to 200 bases, more preferably of the length of 20 to 100 bases, most preferably 40-60 bases. It will be understood that each probe sequence may also comprise linker sequences in addition to the sequence that is complementary to its target sequence. As used herein, a linker sequence refers to a sequence between the sequence that is complementary to its target sequence and the surface of support. For example, the profiling arrays can comprise one probe specific to each target gene or exon. However, if desired, the profiling arrays may contain at least 2, 5, 10, 100, 1000 probes specific to some target genes or exons. For example, the array may contain probes tiled across the sequence of the longest mRNA isoform of a gene at single base steps.

For example, cDNAs from cell samples from two different conditions are hybridized to the binding sites of the microarray using a two-color protocol. In the case of a comparison between cells treated with siRNA molecules versus the same type of cells that are not treated with siRNA molecules, one cell sample is exposed to the siRNA and another cell sample of the same type is not exposed to the siRNA. The cDNA derived from each of the two treatments are differently labeled (e.g., with Cy3 and Cy5) so that they can be distinguished. In one embodiment, for example, cDNA from a cell treated with an siRNA is synthesized using a fluorescein-labeled dNTP, and cDNA from a second cell, not siRNA-exposed, is synthesized using a rhodamine-labeled dNTP. When the two cDNAs are mixed and hybridized to the microarray, the relative intensity of signal from each cDNA set is determined for each site on the array, and any relative difference in abundance of a particular gene detected.

In the example described above, the cDNA from the siRNA-treated cell will fluoresce green when the fluorophore is stimulated and the cDNA from the untreated cell will fluoresce red. As a result, when the siRNA treatment has no effect, either directly or indirectly, on the transcription and/or post-transcriptional splicing of a particular gene in a cell, the gene and/or exon expression patterns will be indistinguishable in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the siRNA-exposed cell is treated with a siRNA that, directly or indirectly, change the transcription and/or post-transcriptional splicing of a particular gene in the cell, the gene and/or exon expression pattern as represented by ratio of green to red fluorescence for each gene or exon binding site will change. When the siRNA increases the prevalence of an mRNA, the ratios for each gene or exon expressed in the mRNA will increase, whereas when the siRNA decreases the prevalence of an mRNA, the ratio for each gene or exons expressed in the mRNA will decrease.

The use of a two-color fluorescence labeling and detection scheme to define alterations in gene expression has been described in connection with detection of mRNAs, e.g., in Shena et al., “Quantitative monitoring of gene expression patterns with a complementary DNA microarray,” Science 270:467-470, 1995, which is incorporated by reference in its entirety for all purposes. The scheme is equally applicable to labeling and detection of genes or exons. An advantage of using cDNA labeled with two different fluorophores is that a direct and internally controlled comparison of the mRNA or exon expression levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular gene or exon in, e.g., an siRNA-treated and an untreated cell. Furthermore, labeling with more than two colors is also contemplated in the present invention. In some embodiments of the invention, at least 5, 10, 20, or 100 dyes of different colors can be used for labeling. Such labeling permits simultaneous hybridizing of the distinguishably labeled cDNA populations to the same array, and thus measuring, and optionally comparing the expression levels of, mRNA molecules derived from more than two samples. Dyes that can be used include, but are not limited to, fluorescein and its derivatives, rhodamine and its derivatives, texas red, 5′carboxy-fluorescein (“FMA”), 2′,7′-dimethoxy-4′,5′-dichloro-6-carboxy-fluorescein (“JOE”), N,N,N′,N′-tetramethyl-6-carboxy-rhodamine (“TAMRA”), 6′carboxy-X-rhodamine (“ROX”), HEX, TET, IRD40, and IRD41, cyamine dyes, including but are not limited to Cy3, Cy3.5 and Cy5; BODIPY dyes including but are not limited to BODIPY-FL, BODIPY-TR, BODIPY-TMR, BODIPY-630/650, and BODIPY-650/670; and ALEXA dyes, including but are not limited to ALEXA-488, ALEXA-532, ALEXA-546, ALEXA-568, and ALEXA-594; as well as other fluorescent dyes which will be known to those who are skilled in the art.

Hybridization data may be measured at a plurality of different hybridization times so that the evolution of hybridization levels to equilibrium can be determined. In such embodiments, hybridization levels are most preferably measured at hybridization times spanning the range from 0 to in excess of what is required for sampling of the bound polynucleotides (i.e., the probe or probes) by the labeled polynucleotides so that the mixture is close to or substantially reached equilibrium, and duplexes are at concentrations dependent on affinity and abundance rather than diffusion. However, the hybridization times are preferably short enough that irreversible binding interactions between the labeled polynucleotide and the probes and/or the surface do not occur, or are at least limited. For example, in embodiments wherein polynucleotide arrays are used to probe a complex mixture of fragmented polynucleotides, typical hybridization times may be approximately 0-72 hours. Appropriate hybridization times for other embodiments will depend on the particular polynucleotide sequences and probes used, and may be determined by those skilled in the art (see, e.g., Sambrook et al., eds., 1989, Molecular Cloning: A Laboratory Manual, 2nd ed., Vol. 1-3, Cold Spring Harbor Laboratory, Cold Spring Harbor, N.Y.).

In one embodiment, hybridization levels at different hybridization times are measured separately on different, identical microarrays. For each such measurement, at the hybridization time when hybridization level is measured, the microarray is washed briefly, preferably in room temperature in an aqueous solution of high to moderate salt concentration (e.g., 0.5 to 3 M salt concentration) under conditions which retain all bound or hybridized polynucleotides while removing all unbound polynucleotides. The detectable label on the remaining, hybridized polynucleotide molecules on each probe is then measured by a method which is appropriate to the particular labeling method used. The resulting hybridization levels are then combined to form a hybridization curve. In another embodiment, hybridization levels are measured in real time using a single microarray. In this embodiment, the microarray is allowed to hybridize to the sample without interruption and the microarray is interrogated at each hybridization time in a non-invasive manner. In still another embodiment, a single array is hybridized for a short time, washed and the amount of hybridization of each gene is measured, the array is again hybridized with the same sample, washed and the amount of hybridization is measured again to obtain the hybridization time curve.

Preferably, at least two hybridization levels at two different hybridization times are measured, a first one at a hybridization time that is close to the time scale of cross-hybridization equilibrium and a second one measured at a hybridization time that is longer than the first one. The time scale of cross-hybridization equilibrium depends, inter alia, on sample composition and probe sequence and may be determined by one skilled in the art. In preferred embodiments, the first hybridization level is measured at between 1 to 10 hours, whereas the second hybridization time is measured at about 2, 4, 6, 10, 12, 16, 18, 48 or 72 times as long as the first hybridization time.

Preparing Probes for Microarrays: As noted above, the “probe” to which a particular polynucleotide molecule, such as a gene or exon, specifically hybridizes according to the invention is a complementary polynucleotide sequence. Preferably one or more probes are selected for each target gene or exon. For example, when a minimum number of probes are to be used for the detection of a gene or exon, the probes normally comprise nucleotide sequences greater than about 40 bases in length. Alternatively, when a large set of redundant probes is to be used for a gene or exon, the probes normally comprise nucleotide sequences of about 40-60 bases. The probes can also comprise sequences complementary to full length exons. The lengths of exons can range from less than 50 bases to more than 200 bases. Therefore, when a probe length longer than exon is to be used, it is preferable to augment the exon sequence with adjacent constitutively spliced exon sequences such that the probe sequence is complementary to the continuous mRNA fragment that contains the target exon. This will allow comparable hybridization stringency among the probes of an exon profiling array. It will be understood that each probe sequence may also comprise linker sequences in addition to the sequence that is complementary to its target sequence.

The probes may comprise DNA or DNA “mimics” (e.g., derivatives and analogues) corresponding to a portion of a gene or an exon of a gene in an organism's genome. In one embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of gene or exon segments from genomic DNA, cDNA (e.g., by RT-PCR), or cloned sequences. PCR primers are preferably chosen based on known sequence of the genes or exons or cDNA that result in amplification of unique fragments (i.e., fragments that do not share more than 10 bases of contiguous identical sequence with any other fragment on the microarray). Computer programs that are well known in the art are useful in the design of primers with the required specificity and optimal amplification properties, such as Oligo version 5.0 (National Biosciences). Typically each probe on the microarray will be between 20 bases and 600 bases, and usually between 30 and 200 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g. using N-phosphonate or phosphoramidite chemistries (Froehler et al., Nucleic Acid Res. 14:5399-5407, 1986; McBride et al., Tetrahedron Lett. 24:246-248, 1983). Synthetic sequences are typically between about 15 and about 600 bases in length, more typically between about 20 and about 100 bases, most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568, 1993; U.S. Pat. No. 5,539,083).

In other embodiments, the hybridization sites (i.e., the probes) are made from plasmid or phage clones of genes, cDNAs (e.g., expressed sequence tags), or inserts therefrom (Nguyen et al., Genomics 29:207-209, 1995).

Attaching Probes to the Solid Surface to Form an Array: Polynucleotide probes can be deposited on a support to form the array. Polynucleotide probes can also be synthesized directly on the support to form the array. The probes are attached to a solid support or surface, which may be made, e.g., from glass, plastic (e.g., polypropylene, nylon), polyacrylamide, nitrocellulose, gel, or other porous or nonporous material.

One method for attaching the nucleic acids to a surface is by printing on glass plates, as is described generally by Schena et al., Science 270:467-470, 1995. This method is especially useful for preparing microarrays of cDNA (See also, DeRisi et al., Nature Genetics 14:457-460, 1996; Shalon et al., Genome Res. 6:639-645, 1996; and Schena et al., Proc. Natl. Acad. Sci. U.S.A. 93:10539-11286, 1995).

Another method for making microarrays is by making high-density polynucleotide arrays. Techniques are known for producing arrays containing thousands of oligonucleotides complementary to defined sequences, at defined locations on a surface using photolithographic techniques for synthesis in situ (see, Fodor et al., Science 251:767-773, 1991; Pease et al., Proc. Natl. Acad. Sci. U.S.A. 91:5022-5026, 1994; Lockhart et al., Nature Biotechnology 14:1675, 1996; U.S. Pat. Nos. 5,578,832; 5,556,752; and 5,510,270) or other methods for rapid synthesis and deposition of defined oligonucleotides (Blanchard et al., Biosensors & Bioelectronics 11:687-690 (1996)). When these methods are used, oligonucleotides (e.g., 60-mers) of known sequence are synthesized directly on a surface such as a derivatized glass slide. The array produced can be redundant, with several polynucleotide molecules per gene or exon.

Other methods for making microarrays, e.g., by masking (Maskos and Southern, Nucl. Acids. Res. 20:1679-1684, 1992), may also be used. In principle, and as noted supra, any type of array, for example, dot blots on a nylon hybridization membrane (see Sambrook et al., supra) could be used. However, as will be recognized by those skilled in the art, very small arrays will frequently be preferred because hybridization volumes will be smaller.

Microarrays useful in the practice of the invention can be manufactured, for example, by means of an ink jet printing device for oligonucleotide synthesis, e.g., using the methods and systems described by Blanchard in International Patent Publication No. WO 98/41531, published Sep. 24, 1998; Blanchard et al., Biosensors and Bioelectronics 11:687-690, 1996; Blanchard, 1998, in Synthetic DNA Arrays in Genetic Engineering, vol. 20, J. K. Setlow, ed., Plenum Press, New York at pages 111-123; and U.S. Pat. No. 6,028,189 to Blanchard. Specifically, the polynucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Polynucleotide probes are normally attached to the surface covalently at the 3′ end of the polynucleotide. Alternatively, polynucleotide probes can be attached to the surface covalently at the 5′ S end of the polynucleotide (see for example, Blanchard, 1998, in Synthetic DNA Arrays in Genetic Engineering, vol. 20, J. K. Setlow, ed., Plenum Press, New York at pages 111-123).

Target Polynucleotide Molecules: Target polynucleotides which may be analyzed by using microarrays include RNA molecules such as, but by no means limited to messenger RNA (mRNA) molecules, ribosomal RNA (rRNA) molecules, cRNA molecules (i.e., RNA molecules prepared from cDNA molecules that are transcribed in vivo) and fragments thereof. Target polynucleotides which may also be analyzed by using microarrays include, but are not limited to, DNA molecules such as genomic DNA molecules, cDNA molecules, and fragments thereof including oligonucleotides, ESTs, and STSs.

The target polynucleotides may be from any source. For example, the target polynucleotide molecules may be naturally occurring nucleic acid molecules such as genomic or extragenomic DNA molecules isolated from an organism, or RNA molecules, such as mRNA molecules, isolated from an organism. Alternatively, the polynucleotide molecules may be synthesized, including, e.g., nucleic acid molecules synthesized enzymatically in vivo or in vitro, such as cDNA molecules, or polynucleotide molecules synthesized by PCR, RNA molecules synthesized by in vitro transcription, etc. The sample of target polynucleotides can comprise, e.g., molecules of DNA, RNA, or copolymers of DNA and RNA. In preferred embodiments, the target polynucleotides of the invention will correspond to particular genes or to particular gene transcripts (e.g., to particular mRNA sequences expressed in cells or to particular cDNA sequences derived from such mRNA sequences). However, in many embodiments, particularly those embodiments wherein the polynucleotide molecules are derived from mammalian cells, the target polynucleotides may correspond to particular fragments of a gene transcript. For example, the target polynucleotides may correspond to different exons of the same gene, e.g., so that different splice variants of that gene may be detected and/or analyzed.

In preferred embodiments, the target polynucleotides to be analyzed are prepared in vitro from nucleic acids extracted from cells. For example, in one embodiment, RNA is extracted from cells (e.g., total cellular RNA, poly(A)⁺ messenger RNA, or a fraction thereof) and messenger RNA is purified from the total extracted RNA. Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation and an oligo dT purification (Chirgwin et al., Biochemistry 18:5294-5299, 1979). In another embodiment, RNA is extracted from cells using guanidinium thiocyanate lysis followed by purification on RNeasy columns (Qiagen). cDNA is then synthesized from the purified mRNA using, e.g., oligo-dT or random primers. In preferred embodiments, the target polynucleotides are cRNA prepared from purified messenger RNA extracted from cells. As used herein, cRNA is defined here as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997; see also, U.S. patent application Ser. No. 09/411,074, filed Oct. 4, 1999, by Linsley and Schelter and PCT publication No. WO 02/44399). Both oligo-dT primers (U.S. Pat. Nos. 5,545,522 and 6,132,997) or random primers (PCT publication No. WO 02/44399) that contain an RNA polymerase promoter or complement thereof can be used. Preferably, the target polynucleotides are short and/or fragmented polynucleotide molecules which are representative of the original nucleic acid population of the cell.

The target polynucleotides to be analyzed by the methods and compositions of the invention are preferably detectably labeled. For example, cDNA can be labeled directly, e.g., with nucleotide analogs, or indirectly, e.g., by making a second, labeled cDNA strand using the first strand as a template. Alternatively, the double-stranded cDNA can be transcribed into cRNA and labeled.

Preferably, the detectable label is a fluorescent label, e.g., by incorporation of nucleotide analogs. Other labels suitable for use in the present invention include, but are not limited to, biotin, imminobiotin, antigens, cofactors, dinitrophenol, lipoic acid, olefinic compounds, detectable polypeptides, electron rich molecules, enzymes capable of generating a detectable signal by action upon a substrate, and radioactive isotopes. Preferred radioactive isotopes include ³²P, ³⁵S, ¹⁴C, ¹⁵N and ¹²⁵I. Fluorescent molecules suitable for the present invention include, but are not limited to, fluorescein and its derivatives, rhodamine and its derivatives, texas red, 5′carboxy-fluorescein (“FMA”), 2′,7′-dimethoxy-4′,5′-dichloro-6-carboxy-fluorescein (“JOE”), N,N,N′,N′-tetramethyl-6-carboxy-rhodamine (“TAMRA”), 6′carboxy-X-rhodamine (“ROX”), HEX, TET, IRD40, and IRD41. Fluorescent molecules that are suitable for the invention further include: cyamine dyes, including by not limited to Cy3, Cy3.5 and Cy5; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin, and colloidal gold. Alternatively, in less preferred embodiments the target polynucleotides may be labeled by specifically complexing a first group to the polynucleotide. A second group, covalently linked to an indicator molecules and which has an affinity for the first group, can be used to indirectly detect the target polynucleotide. In such an embodiment, compounds suitable for use as a first group include, but are not limited to, biotin and iminobiotin. Compounds suitable for use as a second group include, but are not limited to, avidin and streptavidin.

Hybridization to Microarrays: As described supra, nucleic acid hybridization and wash conditions are chosen so that the polynucleotide molecules to be analyzed (referred to herein as the “target polynucleotide molecules”) specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93:10614). Useful hybridization conditions are also provided in, e.g., Tijessen, 1993, Hybridization With Nucleic Acid Probes, Elsevier Science Publishers B.V. and Kricka, 1992, Nonisotopic DNA Probe Techniques, Academic Press, San Diego, Calif.

Particularly preferred hybridization conditions include hybridization at a temperature at or near the mean melting temperature of the probes (e.g., within 5° C., more preferably within 2° C.) in 1 M NaCl, 50 mM MES buffer (pH 6.5), 0.5% sodium Sarcosine and 30% formamide.

Signal Detection and Data Analysis: It will be appreciated that when target sequences, e.g., CDNA or cRNA, complementary to the RNA of a cell are made and hybridized to a microarray under suitable hybridization conditions, the level of hybridization to the site in the array corresponding to a gene or an exon of any particular gene will reflect the prevalence in the cell of mRNA or mRNAs transcribed from the exon of that gene. For example, when detectably labeled cDNA complementary to total cellular mRNA is hybridized to a microarray, the site on the array corresponding to an exon of a gene that is not transcribed, or is removed during RNA splicing, in the cell will have little or no signal, and an exon of a gene that is transcribed will have a relatively strong signal. The relative abundance of different mRNAs produced from the same gene by alternative splicing is then determined by the signal strength pattern across the whole set of exons monitored for the gene.

In preferred embodiments, target sequences, e.g., cDNAs or cRNAs, from two different cells are hybridized to the binding sites of the microarray. For example, one cell sample is exposed to an siRNA and another cell sample of the same type is not exposed to the siRNA. The cDNA or cRNA derived from each of the two samples are differently labeled so that they can be distinguished. In one embodiment, for example, cDNA from a cell treated with an siRNA is synthesized using a fluorescein-labeled dNTP, and cDNA from a second cell, not siRNA-exposed, is synthesized using a rhodamine-labeled dNTP. When the two cDNAs are mixed and hybridized to the microarray, the relative intensity of signal from each cDNA set is determined for each site on the array, and any relative difference in abundance of a particular exon detected.

In the example described above, the cDNA from the siRNA-treated cell will fluoresce green when the fluorophore is stimulated and the cDNA from the untreated cell will fluoresce red. As a result, when the siRNA treatment has no effect, either directly or indirectly, on the transcription and/or post-transcriptional splicing of a particular gene in a cell, the gene or exon expression patterns will be indistinguishable in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the siRNA-exposed cell is treated with an siRNA that, directly or indirectly, changes the transcription and/or post-transcriptional splicing of a particular gene in the cell, the gene or exon expression pattern as represented by ratio of green to red fluorescence for each gene or exon binding site will change. When the siRNA increases the prevalence of an mRNA, the ratios for each gene or exon expressed in the mRNA will increase, whereas when the siRNA decreases the prevalence of an mRNA, the ratio for each gene or exon expressed in the mRNA will decrease.

The use of a two-color fluorescence labeling and detection scheme to define alterations in gene expression has been described in connection with detection of mRNAs, e.g., in Shena et al., Science 270:467-470, 1995, which is incorporated by reference in its entirety for all purposes. The scheme is equally applicable to labeling and detection of exons. An advantage of using target sequences, e.g., cDNAs or cRNAs, labeled with two different fluorophores is that a direct and internally controlled comparison of the mRNA or exon expression levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular gene or exon in, e.g., a siRNA-treated cell and an untreated cell.

Single-channel detection methods, e.g., using one-color fluorescence labeling, may be used (see U.S. provisional patent application Ser. No. 60/227,966, filed on Aug. 25, 2000, and incorporated by reference herein in its entirety). In this embodiment, arrays comprising reverse-complement (RC) probes are designed and produced. Because a reverse complement of a DNA sequence has sequence complexity that is equivalent to the corresponding forward-strand. (FS) probe that is complementary to a target sequence with respect to a variety of measures (e.g., measures such as GC content and GC trend are invariant under the reverse complement), an RC probe is used as a control probe for determination of the level of non-specific cross hybridization to the corresponding FS probe. The significance of the FS probe intensity of a target sequence is determined by comparing the raw intensity measurement for the FS probe and the corresponding raw intensity measurement for the RC probe in conjunction with the respective measurement errors. In a preferred embodiment, a gene or exon is considered to be present if the intensity difference between the FS probe and the corresponding RC probe is significant. More preferably, a gene or exon is considered to be present if the FS probe intensity is also significantly above background level. Single-channel detection methods can be used in conjunction with multi-color labeling. In one embodiment, a plurality of different samples, each labeled with a different color, is hybridized to an array. Differences between FS and RC probes for each color are used to determine the level of hybridization of the corresponding sample.

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., Genome Res. 6:639-645, 1996). The arrays can be scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., Genome Res. 6:639-645, 1996. Alternatively, the fiber-optic bundle described by Ferguson et al., Nature Biotech. 14:1681-1684, 1996, may be used to monitor mRNA abundance levels at a large number of sites simultaneously.

Signals are recorded and can be analyzed by computer, e.g., using a 12-bit analog to digital board. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by siRNA transfection, gene deletion, or any other tested event.

Comparison of Gene Expression Levels: Art-recognized statistical techniques can be used to compare the levels of expression of individual genes in order to identify genes which have an expression pattern that correlates with a response of living cells of a cell type to an agent. For example, a t-test can be used to determine whether the mean value of repeated measurements of the level of expression of a particular gene is significantly different in a cell treated with siRNA and contacted with an agent, compared to a cell that has not been treated with siRNA and which is contacted with the same agent.

The following publications describe examples of art-recognized techniques that can be used to compare the levels of expression of individual genes in treated and untreated mammalian cells to identify genes which exhibit significantly different expression levels: Nature Genetics, Vol. 32, ps. 461-552 (supplement December 2002); Bioinformatics 18 (4):546-54 (April 2002); Dudoit, et al. Technical Report 578, University of California at Berkeley; Tusher et al., Proc. Nat'l. Acad. Sci. U.S.A. 98 (9):5116-5121 (April 2001); and Kerr, et al., J. Comput. Biol. 7: 819-837.

Representative examples of other statistical tests that are useful in the practice of the present invention include the chi squared test which can be used, for example, to test for association between two factors (e.g., positive or negative correlation of gene expression with the presence of a disease state in a mammalian cell). Again by way of example, art-recognized correlation analysis techniques can be used to test whether a correlation exists between two sets of measurements (e.g., between gene expression and disease state). Standard statistical techniques can be found in statistical texts, such as Modern Elementary Statistics, John E. Freund, 7^{th} edition, published by Prentice-Hall; and Practical Statistics for Environmental and Biological Scientists, John Townend, published by John Wiley & Sons, Ltd.

Methods for confirming that a gene contributes to a response of a living cell to an agent: In a second aspect, the present invention provides methods for confirming that a gene contributes to a response of a living cell to an agent. The methods of this aspect of the invention each include the steps of (a) functionally inactivating a gene in a living cell of a cell type by introducing interfering RNA molecules into the cell, wherein the gene has an expression pattern that correlates with a response of living cells of the cell type to an agent, and wherein a copy of the gene is present in all living cells of the cell type; and (b) confirming that the gene contributes to the response of the living cell to the agent by determining that the response of the living cell to the agent is different from the response of a living cell, that does not comprise the interfering RNA molecules, of the cell type to the agent.

The teachings of the present application with respect to the first aspect of the invention, are also applicable to the methods of the second aspect of the invention (e.g., the teachings with respect to functional inactivation of a gene using interfering RNA molecules).

Methods for identifying a mammalian subject responsive to a KSP inhibitor:

In a third aspect, the present invention provides methods for identifying a mammalian subject responsive to a KSP inhibitor. These methods each include the steps of analyzing chromosome 20 from cancerous cells of a mammalian subject to determine whether portion 20q of chromosome 20 is amplified in the cancerous cells, wherein the mammalian subject is identified as being responsive to a KSP inhibitor provided that portion 20q of chromosome 20 is not amplified in the cancerous cells.

Any useful method can be used to determine whether portion 20q of chromosome 20 is amplified in the cancerous cells. For example, comparative genomic hybridization (CGH) can be used to determine whether portion 20q of chromosome 20 is amplified in the cancerous cells. Exemplary CGH methods are described in the following publications which are each incorporated herein by reference in their entirety: Karhu R, et al., “Quality control of CGH: impact of metaphase chromosomes and the dynamic range of hybridization” Cytometry 28: 198-205 (1997); and Tirkkonen M, et al., “Molecular cytogenetics of primary breast cancer by CGH”, Genes Chromosomes Cancer 21: 177-184 (1998).

The following examples merely illustrate the best mode now contemplated for practicing the invention, but should not be construed to limit the invention.

### EXAMPLE 1

This Example describes a representative method for determining whether genes STK 6 and TPX 2 mediate resistance of human colon cancer cell lines to the KSP inhibitor L′962 ((1S)-1-{[(2S)-4-(2,5-difluorophenyl)-2-phenyl-2,5-dihydro-1H-pyrrol-1-yl]carbonyl}-2-methylpropylamine) which is a putative anti-cancer agent.

Twenty-four human colon cancer cell lines were analyzed in vitro to determine their dose response characteristics with respect to KSP inhibitor L′962. The identity and ATCC accession number of the 24 cell lines (and 10 reference cell lines) are set forth in Table 1.

TABLE 1

| Cell line | ATCC number | Culture Medium |
|---|---|---|
| SW620 | CCL-227 | DMEM |
| HT-29 | HTB-38 | DMEM |
| SW480 | CCL-228 | DMEM |
| WiDr | CCL-218 | DMEM |
| Caco-2 | HTB-37 | DMEM |
| SW837 | CCL-235 | DMEM |
| LS1034 | CRL-2158 | DMEM |
| LS123 | CCL-255 | DMEM |
| SW1116 | CCL-233 | DMEM |
| SW948 | CCL-237 | DMEM |
| HCT116 | CCL-247 | DMEM |
| SW48 | CCL-231 | DMBM |
| DLD-1 | CCL-221 | DMEM |
| LoVo | CCL-229 | DMEM |
| HCT-15 | CCL-225 | DMEM |
| LS174T | CL-188 | DMEM |
| RKO | CRL-2577 | DMEM |
| SW403 | CCL-230 | DMEM |
| SW1417 | CCL-238 | DMEM |
| HCT-8 | CCL-244 | RPMI |
| T84 | CCL-.248 | DMEM |
| LSl8O | CL-187 | DMEM |
| RKO-AS45-1 | CRL -2579 | DMEM |
| SW1463 | CCL-234 | DMEM |

| Reference pool | |
|---|---|
| HCT116 | CCL-247 |
| SW48 | CCL-231 |
| DLD-1 | CCL-221 |
| HCT-15 | CCL-225 |
| LS174T | CL-188 |
| HT-29 | HTB-38 |
| SW480 | CCL-228 |
| SW837 | CCL-235 |
| LS1034 | CRL-2158 |
| SW620 | CCL-227 |

The colon cancer cell lines were maintained in DMEM plus 10% FBS, or in RPMI (plus 10% FBS, penicillin/streptomycin, 10 mM HEPES and 1 mM sodium pyruvate). Cells were seeded into 96-well plates at a density of 1500 or 3000 cells/well. 24 hours after seeding, cells were treated with L′962 in the same medium. Control cells received DMSO instead of L′962. 72 hours following treatment with L′962, cell survival was measured by Alamar blue assay, corrected for background (no cells.) Cell response (survival) in the presence of L′962 was determined as a percentage of control cell growth. The level of cell survival was lower in cell lines that are sensitive to L′962 than in cell lines that are resistant to L′962.

With respect to gene expression profiling, colon cancer cell lines were maintained in DMEM plus 10% FBS, or RPMI plus 10% FBS. Cells were seeded into T75 flasks and cultured until confluence was reached at 70-80%. Cells were harvested and total RNA extracted using an RNeasy kit (Qiagen) and processed for hybridization as described previously. (Hughes, T. R. and Mao, M, Nature Biotech., 19: 342-347 (2001)). RNA from each individual cell line was hybridized against a reference pool of RNA to Agilent microarrays containing oligonucleotides corresponding to approximately 21,000 human genes. Ratio hybridizations were performed with fluorescent label reversal to eliminate dye bias. Microarrays were either purchased from Agilent Technologies or synthesized as described (Hughes, T. R. and Mao, M, supra). Error models have been described previously (Id). The reference pool consisted of RNA from 10 colon cancer cell lines. (see Table 1).

The percentage values for control cell growth at the highest 3 doses of L′962 were averaged to provide the value for the maximum amount of cell growth inhibition for L′962 in each cell line. The EC50 for each cell line was calculated by analyzing the growth versus dose curve using GraphPad Prism 4 software (GraphPad Software Inc.). Based on the EC50 values it was observed that the cell lines segregated into two clearly distinguishable populations differing in sensitivity to L′962: one population that was more sensitive to L′962, and one population that was less sensitive to L′962.

There were approximately 21,000 genes on the microarray. Genes were initially selected that were differentially expressed (p<0.01 by the platform error model) in more than 3 cell lines out of a pool of 10 of the 24 cancer cell lines. This procedure excluded genes that were differentially expressed between different cancer cell lines, and which might otherwise be mistakenly identified as being expressed in response to treatment of the cell lines with L′962. The correlation of expression level [log(ratio)] with the maximum growth inhibition, or log(EC50), across all cell lines, was calculated for each of the genes selected in this manner, using the Pearson correlation coefficient. Those genes with a magnitude of correlation greater than 0.5 were selected as reporters for responsiveness to L′962. 468 of these reporter genes were positively correlated (i.e., were more highly expressed in L′962-resistant cell lines), and 820 genes were negatively correlated (i.e., were expressed at a lower level in L′962-resistant cell lines).

The reporter genes identified in this manner were validated using a leave-one-out cross-validation approach which included the following steps. First, one sample was left out, and reporter genes were selected using the remaining samples (or remaining training samples, as described herein). The reporter genes were divided into the following three sub-groups: reporters negatively correlated with the maximum growth inhibition or log(EC50); reporters positively correlated with the maximum growth inhibition or log(EC50), and which are also located on chromosome 20q; and reporters positively correlated with the maximum growth inhibition or log(EC50), but which are not located on chromosome 20q. The mean log(ratio) of each sub-group of reporters in each cell line were then calculated, and a linear fit was performed between the mean log(ratio) and the maximum growth inhibition, or log(EC50), based on the remaining samples (or remaining training samples). The fitting parameter was used to predict the left-out sample, by each of the 3 sub-groups of reporters, respectively. The foregoing steps were repeated to leave each sample out once. Finally, evaluate the predictive power of each group of genes by calculating the correlation between measured and predicted maximum growth inhibition and log(EC50).

With respect to the selection of training samples, for EC50 reporter selection, the training samples were further limited by an extra-layer of leave-one-out-cross-validation (LOOCV) as follows. Using all the samples, the LOOCV process was used to select reporters and predict the left out sample. The prediction were repeated using three subset of reporters, namely reporters negatively correlated with log(EC50), reporters positively correlated with log(EC50) and which are also on chromosome 20q, and reporters positively correlated with log(EC50) but which are not on 20q. The standard deviation was calculated between the predicted log(EC50) and the measured log(EC50) for three sets of predictions. Samples having standard deviation greater than a value of 1 in two or all three prediction sets were excluded.

This leave-one-out cross-validation process showed that baseline expression predicted the log₁₀EC50 with a correlation of 0.65, and a P-value of 0.02%.

Among the reporter genes positively correlated with resistance, there was a significant enrichment of genes on chromosome 20q. Reporter genes from chromosome 20q had much of the predictive power of the entire set of positively correlated reporters (data not shown). Chromosome 20q is frequently amplified in colon, breast, and ovarian cancers (Hodgson J G, et al., Breast Cancer Res. Treat. 78 (3): 337-45 (2003); Tanner M M, et al., Clin Cancer Res. 6 (5): 1833-9 (2000); Warner S L, et al., Mol. Cancer Ther. 2 (6): 589-95 (2003)). One of the amplified genes located on chromosome 20q that is associated with tumorigenesis is STK6 (Ewart-Toland A, et al., Nat Genet. 34 (4): 403-12 (2003)), a serine/threonine protein kinase that phosphorylates KSP in Xenopus (Giet R, et al., J Biol Chem. 274 (21): 15005-13 (1999)). STK6 is an oncogene that localizes to the centrosome, and its overexpression leads to polyploidy, centrosome amplification, and taxol resistance (Warner S L, et al., Mol Cancer Ther. 2 (6): 589-95 (2003)). STK6 is overexpressed in poor prognosis breast cancer patients (van 't Veer L J, et al., Nature 415 (6871): 530-6 (2002)), and may be amplified in colon tumors (Bischoff J R, et al., EMBO J. 17 (11): 3052-65 (1998)). STK6 mRNA levels were measured in the 24 colon tumor cell lines using quantitative PCR analysis (TaqMan). The levels of STK6 mRNA in these lines were correlated with log₁₀EC50 of L′962 (r˜0.7, data not shown). Thus, there is a positive correlation between an increased level of STK 6 expression and increased resistance to L′962.

Given its role in the KSP pathway, amplification and/or overexpression of STK6 could affect cellular response to KSP inhibitor through a direct or indirect impact on KSP function. Chromosomal amplification is imprecise, however, and may result in collateral amplification of genes adjacent to the driver gene(s). Consequently, siRNA was used to determine whether amplification of STK 6 mediated resistance to L′962.

siRNA molecules directed against the STK 6 gene were one of only three classes of siRNA molecules, from a library of about 800 classes of siRNA molecules directed against about 800 different genes, that sensitized HeLa cells to L′962. The other two classes of siRNA molecules, that sensitized HeLa cells to L′962, were directed against KSP itself, and against TPX 2, another gene in the KSP pathway that promotes STK6 autophosphorylation (Bayliss R, et al., Mol Cell. 12 (4): 851-62 (2003)). Although 17 genes from chromosome 20q were represented in the siRNA library, only functional inactivation of STK 6 and TPX 2 sensitized cells to L′962, thereby supporting the conclusion that enhanced levels of STK 6 gene expression, and/or TPX 2 gene expression, at least partly mediates the resistance of some human cancer cell lines to L′962.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for determining whether a gene mediates a response of a living cell to an agent, the method comprising the steps of:
(a) functionally inactivating a gene in a living cell of a cell type by introducing interfering RNA molecules into the cell, wherein the gene has an expression pattern that correlates with a response of living cells of the cell type to an agent, and wherein a copy of the gene is present in all living cells of the cell type;
(b) contacting the living cell, comprising the interfering RNA molecules, with the agent; and
(c) determining whether the response of the living cell to the agent is different from the response of a living cell, that does not comprise the interfering RNA molecules, of the cell type to the agent, thereby determining whether the gene mediates the response of living cells of the cell type to the agent.

2. The method of claim 1 wherein the living cell is a mammalian cell.

3. The method of claim 1 wherein the living cell is a plant cell.

4. The method of claim 1 wherein the living cell is a nematode cell.

5. The method of claim 1 wherein the living cell is a Drosophila cell.

6. The method of claim 1 wherein the living cell is a human cell.

7. The method of claim 1 wherein the agent is a chemical agent.

8. The method of claim 1 wherein the agent is an energetic agent.

9. The method of claim 1 wherein the agent is a virus.

10. The method of claim 1 wherein the interfering RNA molecules consist essentially of siRNA molecules.

11. The method of claim 1 wherein the interfering RNA molecules consist essentially of shRNA molecules.

12. The method of claim 1 wherein the interfering RNA molecules consist essentially of long double-stranded RNA molecules.

13. The method of claim 1 wherein the gene is an STK6 gene that is at least 90% identical to the nucleic acid sequence set forth in SEQ ID NO:2.

14. The method of claim 1 wherein the gene is a TPX2 gene that is at least 90% identical to the nucleic acid sequence set forth in SEQ ID NO:3.

15. The method of claim 1 wherein the correlation is a positive correlation.

16. The method of claim 1 wherein the correlation is a negative correlation.

17. The method of claim 1 wherein the living cell, comprising the interfering RNA molecules, is cultured in vitro during functional inactivation of the gene and while the living cell is being contacted with the agent.

18. The method of claim 1 wherein a determination that the gene mediates the response of the living cells of the cell type to the agent is made provided that the response of the living cell, that comprises the interfering RNA molecules, to the agent is different from the response of the living cell, that does not comprise the interfering RNA molecules, to the agent.

19. The method of claim 1 wherein a determination that the gene does not mediate the response of the living cells of the cell type to the agent is made provided that the response of the living cell, that comprises the interfering RNA molecules, to the agent is not different from the response of the living cell, that does not comprise the interfering RNA molecules, to the agent.

20. The method of claim 1 wherein the agent is an inhibitor of a KSP protein.

21. The method of claim 1 further comprising the step of identifying the gene, that has an expression pattern that correlates with a response of living cells of the cell type to the agent, before functionally inactivating the gene using the interfering RNA molecules.

22. The method of claim 21 wherein the gene expression pattern is determined by using a nucleic acid microarray.

23. The method of claim 21 wherein the gene expression pattern is determined by comparing the amount of mRNA transcribed from the gene before a living cell is contacted with the agent, with the amount of mRNA transcribed from the gene after the living cell is contacted with the agent.

24. A method to identify a mammalian subject responsive to a KSP inhibitor, said method comprising the step of analyzing chromosome 20 from cancerous cells of a mammalian subject to determine whether portion 20q of chromosome 20 is amplified in the cancerous cells, wherein the mammalian subject is identified as being responsive to a KSP inhibitor provided that portion 20q of chromosome 20 is not amplified in the cancerous cells.

25. A method for confirming that a gene contributes to a response of a living cell to an agent, the method comprising the steps of:
(a) functionally inactivating a gene in a living cell of a cell type by introducing interfering RNA molecules into the cell, wherein the gene has an expression pattern that correlates with a response of living cells of the cell type to an agent, and wherein a copy of the gene is present in all living cells of the cell type; and
(b) confirming that the gene contributes to the response of the living cell to the agent by determining that the response of the living cell to the agent is different from the response of a living cell, that does not comprise the interfering RNA molecules, of the cell type to the agent.


SEQUENCE LISTING (3 sequences)

SEQ ID NO: 1 - protein, 1056 aa, Homo Sapiens
MASQPNSSAKKKEEKGKNIQVVVRCRPFNLAERKASAHSIVECDPVRKEVSVRTGGLADKSSRKTYTFDMVFGASTKQIDVYRSVVCPILDEVIMGYNCTIFAYGQTGTGKTFTMEGERSPNEEYTWEEDPLAGIIPRTLHQIFEKLTDNGTEFSVKVSLLEIYNEELFDLLNPSSDVSERLQMFDDPRNKRGVIIKGLEEITVHNKDEVYQILEKGAAKRTTAATLMNAYSSRSHSVFSVTIHMKETTIDGEELVKIGKLNLVDLAGSENIGRSGAVDKRAREAGNINQSLLTLGRVITALVERTPHVPYRESKLTRILQDSLGGRTRTSIIATISPASLNLEETLSTLEYAHRAKNILNKPEVNQKLTKKALIKEYTEEIERLKRDLAAAREKNGVYISEENFRVMSGKLTVQEEQIVELIEKIGAVEEELNRVTELFMDNKNELDQCKSDLQNKTQELETTQKHLQETKLQLVKEEYITSALESTEEKLHDAASKLLNTVEETTKDVSGLHSKLDRKKAVDQHNAEAQDIFGKNLNSLFNNMEELIKDGSSKQKAMLEVHKTLFGNLLSSSVSALDTITTVALGSLTSIPENVSTHVSQIFNMILKEQSLAAESKTVLQELINVLKTDLLSSLEMILSPTVVSILKINSQLKHIFKTSLTVADKIEDQKKELDGFLSILCNNLHELQENTICSLVESQKQCGNLTEDLKTIKQTHSQELCKLMNLWTERFCALEEKCENIQKPLSSVQENIQQKSKDIVNKMTFHSQKFCADSDGFSQELRNFNQEGTKLVEESVKHSDKLNGNLEKISQETEQRCESLNTRTVYFSEQWVSSLNEREQELHNLLEVVSQCCEASSSDITEKSDGRKAAHEKQHNIFLDQMTIDEDKLIAQNLELNETIKIGLTKLNCFLEQDLKLDIPTGTTPQRKSYLYPSTLVRTEPREHLLDQLKRKQPELLMMLNCSENNKEETIPDVDVEEAVLGQYTEEPLSQEPSVDAGVDCSSIGGVPFFQHKKSHGKDKENRGINTLERSKVEETTEHLVTKSRLPLRAQINL

SEQ ID NO: 2 - DNA, 2346 bp, Homo Sapiens
acaaggcagcctcgctcgagcgcaggccaatcggctttctagctagagggtttaactcctatttaaaaagaagaacctttgaattctaacggctgagctcttggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagaggcatcatggaccgatctaaagaaaactgcatttcaggacctgttaaggctacagctccagttggaggtccaaaacgtgttctcgtgactcagcaatttccttgtcagaatccattacctgtaaatagtggccaggctcagcgggtcttgtgtccttcaaattcttcccagcgcattcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtctaaaaaaa

SEQ ID NO: 3 - DNA, 3685 bp, Homo Sapiens
agtggactcacgcaggcgcaggagactacacttcccaggaactccgggccgcgttgttcgctggtacctccttctgacttccggtattgctgcggtctgtagggccaatcgggagcctggaattgctttcccggcgctctgattggtgcattcgactaggctgcctgggttcaaaatttcaacgatactgaatgagtcccgcggcgggttggctcgcgcttcgttgtcagatctgaggcgaggctaggtgagccgtgggaagaaaagagggagcagctagggcgcgggtctccctcctcccggagtttggaacggctgaagttcaccttccagcccctagcgccgttcgcgccgctaggcctggcttctgaggcggttgcggtgctcggtcgccgcctaggcggggcagggtgcgagcaggggcttcgggccacgcttctcttggcgacaggattttgctgtgaagtccgtccgggaaacggaggaaaaaaagagttgcgggaggctgtcggctaataacggttcttgatacatatttgccagacttcaagatttcagaaaaggggtgaaagagaagattgcaactttgagtcagacctgtaggcctgatagactgattaaaccacagaaggtgacctgctgagaaaagtggtacaaatactgggaaaaacctgctcttctgcgttaagtgggagacaatgtcacaagttaaaagctcttattcctatgatgccccctcggatttcatcaatttttcatccttggatgatgaaggagatactcaaaacatagattcatggtttgaggagaaggccaatttggagaataagttactggggaagaatggaactggagggctttttcagggcaaaactcctttgagaaaggctaatcttcagcaagctattgtcacacctttgaaaccagttgacaacacttactacaaagaggcagaaaaagaaaatcttgtggaacaatccattccgtcaaatgcttgttcttccctggaagttgaggcagccatatcaagaaaaactccagcccagcctcagagaagatctcttaggctttctgctcagaaggatttggaacagaaagaaaagcatcatgtaaaaatgaaagccaagagatgtgccactcctgtaatcatcgatgaaattctaccctctaagaaaatgaaagtttctaacaacaaaaagaagccagaggaagaaggcagtgctcatcaagatactgctgaaaagaatgcatcttccccagagaaagccaagggtagacatactgtgccttgtatgccacctgcaaagcagaagtttctaaaaagtactgaggagcaagagctggagaagagtatgaaaatgcagcaagaggtggtggagatgcggaaaaagaatgaagaattcaagaaacttgctctggctggaatagggcaacctgtgaagaaatcagtgagccaggtcaccaaatcagttgacttccacttccgcacagatgagcgaatcaaacaacatcctaagaaccaggaggaatataaggaagtgaactttacatctgaactacgaaagcatccttcatctcctgcccgagtgactaagggatgtaccattgttaagcctttcaacctgtcccaaggaaagaaaagaacatttgatgaaacagtttctacatatgtgccccttgcacagcaagttgaagacttccataaacgaacccctaacagatatcatttgaggagcaagaaggatgatattaacctgttaccctccaaatcttctgtgaccaagatttgcagagacccacagactcctgtactgcaaaccaaacaccgtgcacgggctgtgacctgcaaaagtacagcagagctggaggctgaggagctcgagaaattgcaacaatacaaattcaaagcacgtgaacttgatcccagaatacttgaaggtgggcccatcttgcccaagaaaccacctgtgaaaccacccaccgagcctattggctttgatttggaaattgagaaaagaatccaggagcgagaatcaaagaagaaaacagaggatgaacactttgaatttcattccagaccttgccctactaagattttggaagatgttgtgggtgttcctgaaaagaaggtacttccaatcaccgtccccaagtcaccagcctttgcattgaagaacagaattcgaatgcccaccaaagaagatgaggaagaggacgaaccggtagtgataaaagctcaacctgtgccacattatggggtgccttttaagccccaaatcccagaggcaagaactgtggaaatatgccctttctcgtttgattctcgagacaaagaacgtcagttacagaaggagaagaaaataaaagaactgcagaaaggggaggtgcccaagttcaaggcacttcccttgcctcattttgacaccattaacctgccagagaagaaggtaaagaatgtgacccagattgaacctttctgcttggagactgacagaagaggtgctctgaaggcacagacttggaagcaccagctggaagaagaactgagacagcagaaagaagcagcttgtttcaaggctcgtccaaacaccgtcatctctcaggagccctttgttcccaagaaagagaagaaatcagttgctgagggcctttctggttctctagttcaggaaccttttcagctggctactgagaagagagccaaagagcggcaggagctggagaagagaatggctgaggtagaagcccagaaagcccagcagttggaggaggccagactacaggaggaagagcagaaaaaagaggagctggccaggctacggagagaactggtgcataaggcaaatccaatacgcaagtaccagggtctggagataaagtcaagtgaccagcctctgactgtgcctgtatctcccaaattctccactcgattccactgctaaactcagctgtgagctgcggataccgcccggcaatgggacctgctcttaacctcaaacctaggaccgtcttgctttgtcattgggcatggagagaacccatttctccagacttttacctacccgtgcctgagaaagcatacttgacaactgtggactccagttttgttgagaattgttttcttacattactaaggctaataatgagatgtaactcatgaatgtctcgattagactccatgtagttacttcctttaaaccatcagccggccttttatatgggtcttcactctgactagaatttagtctctgtgtcagcacagtgtaatctctattgctattgccccttacgactctcaccctctccccactttttttaaaaattttaaccagaaaataaagatagttaaatcctaagatagagattaagtcatggtttaaatgaggaacaatcagtaaatcagattctgtcctcttctctgcataccgtgaatttatagttaaggatccctttgctgtgagggtagaaaacctcaccaactgcaccagtgaggaagaagactgcgtggattcatggggagcctcacagcagccacgcagcaggctctgggtggggctgccgttaaggcacgttctttccttactggtgctgataacaacagggaaccgtgcagtgtgcattttaagacctggcctggaataaatacgttttgtctttccctcaaaaaaaaaaaaaaaaaaaaaaa